(19) United States
(12) Patent Application Publication
(10) Pub. No.: US 2024/0240023 A1
(43) Pub. Date: Jul. 18, 2024

(54) Cross-linkable Polymers for Fabrication of Biodegradable 3D Hydrogel Scaffold, and Process Thereof

(71) Applicant: Indian Oil Corporation Limited, Mumbai (IN)

(72) Inventors: Mani GAJENDRAN, Faridabad (IN); Jyotiranjan OTA, Faridabad (IN); Samik Kumar HAIT, Faridabad (IN); Sankara Sri Venkata RAMAKUMAR, Faridabad (IN)

(21) Appl. No.: 18/524,167

(22) Filed: Nov. 30, 2023

(30) Foreign Application Priority Data
Jan. 7, 2023 (IN) 202221063505

(51) Int. Cl. C08L 89/06; C08L 67/06; C08L 71/02

(52) U.S. Cl. CPC C08L 89/06; C08L 67/06; C08L 71/02; C08L 2201/06; C08L 2203/02; C08L 2205/05

(57) ABSTRACT

The present invention relates a biodegradable 3D composite hydrogel scaffold and a process for preparing the same from a cross-linkable synthetic biodegradable polymer, methacrylated poly multi-carboxylic acid-polyethylene glycol (MA-PMCA-PEG) and a modified natural polymer maleated gelatin (GEL-MEA). The invention deals with development of cross-linkable synthetic and natural polymers through simple and cost-effective methods. A multicarboxylic acid-b-polyethylene glycol (PMCA-PEG), is synthesized by a direct melt polycondensation technique and thereafter methacrylated using glycidyl methacrylate to prepare cross-linkable MA-PMCA-PEG, which is a new biodegradable material with excellent cross-linking properties. The natural polymer gelatin (GEL) is reacted with maleic anhydride (MEA) to get cross-linkable GEL-MEA.

### FIELD OF THE INVENTION

The present invention relates to a biodegradable 3D composite hydrogel scaffold comprising a cross-linkable synthetic biodegradable polymer methacrylated poly multi-carboxylic acid-polyethylene glycol (MA-PMCA-PEG) and a natural polymer maleated gelatin (GEL-MEA).

More particularly, the present invention relates to a simple and cost-effective development of cross-linkable synthetic and natural polymers for the fabrication of 3D composite biodegradable hydrogels that exhibits anti-viral properties and can be utilized in disinfectant spray or as a coaling matrix for face mask. The present invention deals with production of new cross-linkable polymers and a process to achieve biodegradable hydrogels. A multicarboxylic acid-b-polyethylene glycol (PMCA-PEG), synthesized by a direct melt polycondensation technique is methacrylated using glycidyl methacrylate to prepare cross-linkable methacrylated PMCA-PEG (MA-PMCA-PEG), which is a new biodegradable material with excellent cross-linking properties. The natural polymer gelatin (GEL) is reacted with maleic anhydride (MEA) to get cross-linkable maleated gelatin (GEL-MEA). The approach of using maleated gelatin (GEL-MEA) as a crosslinkable polymer stages a number of advantages over the conventional thiolated crosslinkable polymers. The synthetic polymer MA-PMCA-PEG and modified natural polymer GEL-MEA were cross-linked by using ammonium persulfate and tetramethylethylenediamine (TEMED) to get a distinct 3D hydrogel scaffold. The hydrogel scaffold could be prepared in nano or micro size via water-in-oil (W/O) emulsion method. The nanohydrogel based platform could be used as a carrier for anti-viral nanoparticles in disinfectant spray or as a coating matrix for face mask.

### BACKGROUND OF THE INVENTION

Hydrogel scaffolds used for biomedical applications are usually fabricated by cross-linking functional polymer materials using a cross-linker and initiator. Synthesis of most of the reported cross-linkable polymers involves expensive materials, tedious process, carcinogenic chemicals or non-degradable polymers. The poly (ethylene glycol) diacrylate (PEGDA) has been used as a cross-linkable polymer to fabricate hydrogel scaffolds for soft tissue regeneration (Jeremy, WO, 2006/004951 A2) and drug delivery application (Wu et al., 2010, U.S. Pat. No. 7,670,616B2). The PEGDA based hydrogels are non-cell adhesive; hence biomaterials such as cross linkable collagen, hyaluronic acid, and GEL have been cross-linked with PEGDA to get cell adhesive biocompatible hydrogel. The PEGDA is a non-degradable polymer and very expensive, hence, scaling up of the hydrogel formulation using PEGDA in industrial scale is not viable.

Recently, citric acid-PEG based copolymers received great attention in biomedical field. For instance, Naeini et al., have reported the synthesis and self-assembly of poly(citric acid) (PCA)-block-poly(ethylene glycol) (PEG) copolymer [Nanomedicine: Nanotechnology, Biology, and Medicine 6 (2010) 556-562]. Then, similar citric acid-PEG based polymers were synthesized and used for drug delivery and nanomedicine applications. Naeini et al., have used the PCA-PEG-PCA polymer to synthesize necklace shaped gold NPs [European Polymer Journal 46 (2010) 165-170]. Gajendiran et al., have reported the synthesis and self-aggregation induced emission property of citric acid-PEG hyper branched polymer [Scientific Reports volume 7, Article number: 16418 (2017)], and the same research group has used the citric acid-PEG copolymer for the green synthesis of gold nanoparticles [Journal Material Chemistry B. 2014, 2, 418-427]. However, the cross-linkable methacrylated citric acid-PEG polymer and further cross-linking of the polymer with other natural polymers have not been reported in literature.

Also, several modifications on gelatin with various functional moieties have been performed to make hydrogel scaffold. For instance, Kim et al., have reported hydroxyl phenyl propionic acid modified GEL to get gelatin hydrogel via an enzymatic cross linking (Kim et al., ACS Appl. Bio Mater. 2020, 3, 1646-1655). Kurisawa et al., have disclosed synthesis of hydroxyphenylpropionic acid (HPA) attached gelatin (GEL-HPA) via 1-Ethyl-3-(3-dimethylaminopropyl)carbodiimide (EDC) coupling reaction (Kurisawa et al., WO 2019/226120 A1) Park et al., have disclosed the preparation of hydroxylphenylacetic acid attached gelatin (GHPA) and formation of hydrogel via horseradish peroxidase (FIRP) enzymatic cross-linking reaction (Park et at, EP 2 448 610 B1). The GEL-HPA hydrogel was prepared by using an enzyme HRP as a cross-linker and hydrogen peroxide as a radical initiator. Bulcke et al., have reported methacrylate modified GEL by reacting with methacrylic anhydride to prepare GEL hydrogel via photo cross-linking reaction (Bulcke et al., Biomacromolecules, 2000, 1, 31-38). Munoz et al., have synthesized norbornene attached GEL to prepare GEL hydrogel via an orthogonal thiol-norbomene photochemistry (Monuz et al., Biomater. Sci., 2014, 2, 1063-1072). Bishop et al., have disclosed the cross-linking of GEL using various external cross-linkers such as (1-ethyl-3-3-(dimethylamino)propyl carbodiimide) (EDC) and calcium chloride, but the cross-linking processes were time consuming processes (2 h) (Bishop et al., U.S. Pat. No. 5,834,232).

Thiolation of gelatin is a widely followed strategy to prepare cross-linkable gelatin (Stanley et al., U.S. Pat. Appl. Publ. 2014, US 20140315805 A1 20141023). The thiolated gelatin could be cross-linked with PEGDA to prepare 3D hydrogel scaffold for biomedical application (Sungjun et al, Tissue Eng. Regen. Med. 2022, 19, 309-319).

However, the thiolated gelatin is highly reactive and involves in self cross-linking in an uncontrolled manner under atmospheric air. Hence, the shelf life of the product is a great hindrance for wide commercial use as it may not be useful for making hydrogel after exposure to atmospheric air. Hence, production of the thiolated gelatin based hydrogel in industrial scale is a difficult task. A new breed of material devoid of thiolated gelatin would be helpful to overcome this technical difficulty without compromising the gelation property.

Several cross-linkable polymers have been reported in literature for the fabrication of hydrogel scaffolds. Usually, the PEGDA, thiolated gelatin, thiolated alginate, thiolated chitosan, thiol modified heparin and hyaluronan have been used as cross-linkable polymers. The PEGDA is a non-degradable polymer and the thiolating agents are highly toxic (Category II) in nature, and hence they are not environmentally benign materials. The thiolated polymers are highly expensive and hence, a huge investment is required for their production in large scale. Self-stability of the widely used thiolated polymers under atmospheric air is very low due to the oxidation of sulfhydryl group to form disulfide bond, and hence handling the thiolated polymers in industrial scale is not easy. Hence, new types of biodegradable advanced materials need to be developed via cost effective and simple synthetic procedures for the fabrication of hydrogel scaffold in large scale.

Also, synthesis of cross-linkable polymers such as thiolated gelatin, collagen involves expensive reagents such as Traut's reagent and γ-thiobutyrolactone. In addition, after synthesis, the cross-linkable polymers need to be purified by dialysis and followed by freeze drying. The dialysis tube is very expensive to purify the polymers in bulk scale. Hence, synthesis of the conventional cross-linkable polymers in bulk scale is expensive and for commercial application requires higher cost of investment.

Additionally, synthesis of cross-linkable gelatin involves a series of synthetic process steps Vis. i) addition of the thiolating agents to gelatin backbone under inert atmosphere, ii) dialysis and iii) freeze drying. Particularly, in a synthetic process of 1 g scale of thiolated gelatin, the freeze dryer should run for 4 to 5 days continuously to dry the sample completely. Hence, synthesis of the thiolated gelatin is a tedious process to scale up in bulk scale.

Hence, a simple and cost-effective synthetic procedure needs to be developed to synthesize the cross-linkable gelatin in bulk scale for commercial application.

Recently, the maleate moiety has been used as a cross-linking group to fabricate hydrogel. For instance, Sukhlaaied et al, have reported the maleated polyvinyl alcohol and cross-linked with gelatin to prepare the graft polymer hydrogel [Sukhlaaied et al., Polym. Bull. 8, 2016, DOI 10.1007/s00289-016-1609-3]. Ali et. al. has directly polymerized the maleic anhydride on gelatin backbone in the presence of drug molecule to prepare a drug copolymer [Chemistry and Materials Research, Vol. 7, No. 5, 2015].

Moreover, in the reported papers, maleic anhydride was directly polymerized on gelatin, but the —C═C— of maleate molecule was not free for further cross linking. The cross-linkable maleated gelatin with free —C═C— on maleate moiety has not been reported in literature.

### SUMMARY OF THE INVENTION

Accordingly, the present invention provides a biodegradable and environment friendly cross-linkable synthetic polymer methacrylated poly multi-carboxylic acid-polyethylene glycol (MA-PMCA-PEG) and a process for synthesising/preparing the same by simple synthetic methods. The cross-linkable synthetic polymer MA-PMCA-PEG exhibits multiple active sites which could be used for cross-linking with the natural polymers such as gelatin, collagen and chitosan to get three-dimensional hydrogel scaffolds.

In addition, the cost effectively scalable MA-PMCA-PEG polymer could provide both the cross-linking moiety and free carboxylic acid group into the hydrogel network. The cross-linking moiety could be used to fabricate hydrogel scaffold, while the free carboxylic acid group could exhibit anti-viral property. Hence, the polymer could be used to get an ionic hydrogel matrix for antiviral application.

The present invention provides a simple and new and cost-effective process for the development of stable cross-linkable natural polymer maleated gelatin (GEL-MEA) having free —C═C— group for controlled cross-linking, via an in-house developed procedure using less expensive materials such as gelatin, maleic anhydride, water and acetone.

Fabrication of carboxylic acid enriched hybrid composite hydrogel by cross-linking the MA-PMCA-PEG polymer with other cross-linkable natural polymers such as GEL-MEA, which is new of its kind. The cross-linkable polymers could be used to fabricate anti-viral composite hydrogel in industrial scale by a cost-effective approach. The mechanical properties of the fabricated hydrogel could be tuned by varying the composition of MA-PMCA-PEG and GEL-MEA in the hydrogel scaffold.

The present invention provides a biodegradable 3D composite hydrogel scaffold comprising (i) a synthetic biodegradable polymer methacrylated poly multi-carboxylic acid-polyethylene glycol (MA-PMCA-PEG) and (ii) a natural polymer maleated gelatin (GEL-MEA). The present invention also provides a process for the development of biodegradable 3D composite hydrogel comprising (i) Cross-linkable biodegradable and environment friendly synthetic polymer, i.e., MA-PMCA-PEG and (ii) Cross-linkable natural polymer, i.e., GEL-MEA for the fabrication of hydrogel scaffold at large scale. The new biodegradable MA-PMCA-PEG synthetic polymer is developed via cost effective and simple synthetic procedures. The cross-linkable natural polymer, GEL-MEA is synthesized by a simple and controlled synthetic procedure and is highly stable under normal atmospheric condition at room temperature for a minimum period of four months. Composite 3D hydrogel scaffolds are fabricated by cross-linking MA-PMCA-PEG and GEL-MEA The synthesized polymer exhibits both cross-linking moiety and free carboxylic acid group.

The present invention describes two types of cross-linkable polymers: (i) Cross-linkable synthetic polymer, i.e., MA-PMCA-PEG and (ii) Cross-linkable natural polymer, i.e., GEL-MEA.

The cross-linkable synthetic polymer MA-PMCA-PEG is biodegradable and hence it is environmentally friendly.

The cross-linkable natural polymer, GEL-MEA was synthesized by a simple synthetic procedure. A mixture of aqueous and organic solvents was used in the synthesis of GEL-MEA, and the polymer product was recovered just by increasing the organic composition in the reaction mixture. The organic solvent could be recycled by rotary evaporation. Hence, the synthesis procedure for getting GEL-MEA is technically and economically important. The GEL-MEA described in the present invention is highly stable under normal atmospheric condition at room temperature for a minimum period of four months, and the cross-linking reaction with GEL-MEA could be carried by a controlled process.

### OBJECTIVES OF THE PRESENT INVENTION

It is the primary objective of the present invention is to develop a biodegradable hydrogel by cross-linking a synthetic and a natural polymer.

It is one of the main objectives of the present invention is to synthesize the synthetic and natural cross-linkable polymers in cost effective manner with less expensive materials.

It is one of the main objectives of the present invention is to synthesize the synthetic and natural cross-linkable polymers in simple, controlled, and scalable manner.

It is yet another objective of the present invention is to synthesize the polymer with both cross-linking moiety and free carboxylic acid group for anti-viral property.

It is yet another objective of the present invention is to develop a 3D composite hydrogel scaffold using the biodegradable hydrogel.

### BRIEF DESCRIPTION OF THE DRAWINGS

FIG. 1: Illustrates synthesis of cross-linkable MA-PICA-PEG.

FIG. 2: Illustrates synthesis of cross-linkable GEL-MEA.

FIG. 3: Illustrates process for scaling up of MA-PMCA-PEG polymer.

FIG. 4: Illustrates fabrication of 3D hydrogel matrix using the cross-linkable polymers MA-PMCA-PEG and GEL-MEA.

FIG. 5: Illustrates FTIR spectra of (a) PMCA-PEG and (b) MA-PMCA-PEG polymers.

FIG. 6: Illustrates ¹H-NMR spectra of (a) PMCA-PEG and (b) MA-PMCA-PEG recorded in D₂O.

FIG. 7: Illustrates ¹³C-NMR spectra of (a) PPMCA-PEG and (b) MA-PMCA-PEG recorded in D₂O.

FIG. 8: Illustrates ¹H-NMR spectra of (a) gelatin and (b) GEL-MEA recorded in D₂O.

FIG. 9: Illustrates ¹³C-NMR spectra of (e) gelatin and (b) GEL-MEA recorded in D₂O.

FIG. 10: Illustrates DSC thermograms of (a) PMCA-PEG and (b) MA-PMCA-PEG.

FIG. 11: Illustrates (A) Stress Vs Strain plot of compression test and (B) compressive modulus of different hydrogel matrices. HG1, HG2 and HG3 exhibit 0.5, 0.33 and 0.0 ratio of MA-PMCA-PEG/GEL-MEA.

### ABBREVIATIONS

PEG: polyethylene glycol
PMCA: poly multi-citric acid/poly multi-carboxylic acid.
GEL: gelatin
TEMED: tetramethylethylenediamine
MA-PMCA-PEG: methacrylated poly multi-carboxylic acid-polyethylene glycol
APS: ammonium persulfate
GEL-MEA: maleated gelatin
DMF: dimethyl formamide
GMA: glycidyl methacrylate

### DETAILED DESCRIPTION OF THE INVENTION

For the purpose of promoting an understanding of the principles of the invention, reference will now be made to the embodiments in the specific language will be used to describe the same. It will nevertheless be understood that no limitation of the scope of the invention is thereby intended, such alterations and further modifications in the illustrated composition, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The composition, methods, and examples provided herein are illustrative only and not intended to be limiting.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Unless defined otherwise, all technical and scientific terms used herein have the same meaning as commonly understood by one of ordinary skill in the art to which this disclosure belongs.

Although any methods and materials similar or equivalent to those described herein can be used in the practice or testing of the disclosure, the preferred methods, and materials are now described. All publications mentioned herein are incorporated herein by reference.

The terminology and structure employed herein is for describing, teaching, and illuminating some embodiments and their specific features and elements and does not limit, restrict, or reduce the spirit and scope of the invention.

The present invention provides first time a synthesis of methacrylated poly citric acid-b-poly ethylene glycol branched polymer (MA-PMCA-PEG), anew biodegradable material. The MA-PMCA-PEG polymer exhibits both cross-linkable moiety and free carboxylic acid group. Hence, the polymer could be used to get an ionic hydrogel matrix for antiviral application.

The present invention also provides a synthesis of cross-linkable GEL-MEA for the first time by a new procedure, the GEL-MEA is highly stable under atmospheric air, and cross-linking reaction of GEL-MEA could be carried in a controlled manner.

Fabrication of carboxylic acid enriched hybrid composite hydrogel by cross-linking the MA-PMCA-PEG polymer with other cross-linkable natural polymers such as GEL-MEA, which is new of its kind.

Accordingly, the present invention provides a biodegradable hydrogel for 3D composite hydrogel scaffold comprising:
a synthetic biodegradable polymer methacrylated poly multi-carboxylic acid-polyethylene glycol (MA-PMCA-PEG) and
a modified natural polymer maleated gelatin (GEL-MBA).

In one of the features of the present invention, the synthetic biodegradable polymer methacrylated poly multi-carboxylic acid-polyethylene glycol (MA-PMCA-PEG) to the modified natural polymer maleated gelatin (GEL-MEA) has a ratio in a range of 0.33 to 0.5.

In one of the features of the present invention, the synthetic biodegradable polymer methacrylated poly multi-carboxylic acid-polyethylene glycol (MA-PMCA-PEG) to the modified natural polymer maleated gelatin (GEL-MEA) has a ratio of 0.5 exhibit a swelling factor of 8.0 and a compression modulus of 35.1 kPa.

In another feature of the present invention, the synthetic biodegradable polymer methacrylated poly multi-carboxylic acid-polyethylene glycol (MA-PMCA-PEG) to the modified natural polymer maleated gelatin (GEL-MEA) has a ratio of 0.33 exhibit the swelling factor of 6.2, and the compression modulus of 19.3 kPa.

In another feature of the present invention, the biodegradable hydrogel described above is for use in preparing a biodegradable 3D composite hydrogel scaffold.

The present invention also provides a synthetic biodegradable cross linkable polymer:
methacrylated poly multi-carboxylic acid-polyethylene glycol (MA-PMCA-PEG)

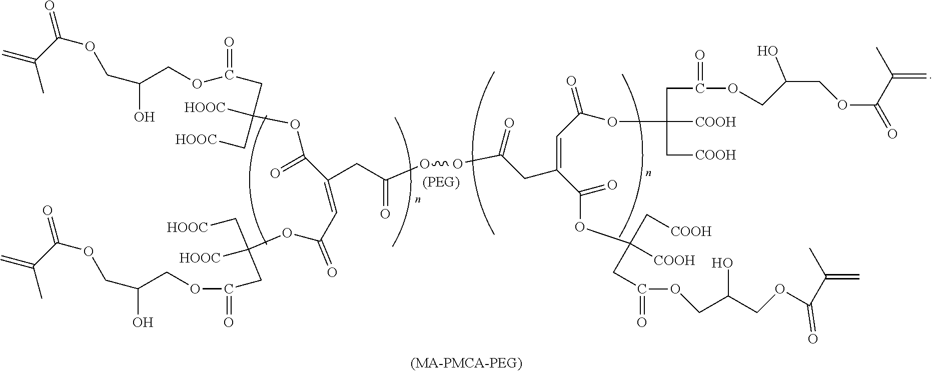

The present invention provides a process for fabricating a biodegradable 3D composite hydrogel scaffold comprising:
preparing a synthetic biodegradable polymer methacrylated poly multi-carboxylic acid-polyethylene glycol (MA-PMCA-PEG);
preparing a modified natural polymer maleated gelatin (GEL-MEA);
mixing of predetermined volume of a solution of the synthetic biodegradable polymer (MA-PMCA-PEG) and a solution of the modified natural polymer (GEL-MEA) to obtain a polymer solution; and
preparing a hydrogel from the polymer solution and molding the hydrogel to obtain the biodegradable 3D composite hydrogel scaffold, wherein different hydrogels are obtained by varying the ratio of (MA-PMC A-PEG) and (GEL-MEA).

In one feature of the present invention, the synthetic biodegradable polymer methacrylated poly multi-carboxylic acid-polyethylene glycol (MA-PMCA-PEG) is prepared by a process comprising reacting poly multi-carboxylic acid-polyethylene glycol (PMCA-PEG) polymer in dimethylformamide (DMF) with glycidyl methacrylate (GMA) in the presence of triethylamine (TEA) and adding diethyl ether to recover MA-PMC A-PEG.

In an exemplary feature of the present invention, the polymer product MA-PMCA-PEG was recovered by drying under rotary evaporation.

In one of the features of the present invention, the glycidyl methacrylate (GMA) is added in a range of 0.02-0.1 mol, and the triethylamine is added in a range of 5-15 mL.

In one of the feature of the present invention, the reaction is carried out at a temperature in a ran_{g}e of 60-80° C. for 15 to 24 h under dark and inert atmosphere with agitation and the solvent is dimethvl forrnanide.

In one of the features of the present invention, the maleation of the natural polymer gelatin using maleic anhydride is described. For maleation of gelatin, a mixture of acetone/water system was used as solvent. After the reaction, the GEL-MEA was precipitated by addition of excess acetone, and at the same time the unreacted maleic anhydride was washed out in acetone. Then, the GEL-MEA was dried by rotary evaporation. The GEL-MEA was synthesized with cost-economic reagents, and it is highly stable under room temperature and atmospheric air.

In one of the features of the present invention, the modified natural polymer maleated gelatin (GEL-MEA) is prepared by a process comprising dissolving gelatin in water to obtain a gelatin solution and reacting the gelatin solution with a maleating agent in acetone to obtain the modified natural polymer maleated gelatin (GEL-MEA).

In one of the features of the present invention, the maleating agent is maleic anhydride; and the amount of gelatin in water is in the range of 5-15%, w/v and the amount of maleic anhydride in acetone is in the range of 40-60%, w/v.

In another feature of the present invention, water is de-ionized distilled water (DDW), In yet another feature of the present invention, in the process for preparing the modified natural polymer mnaleated gelatin (GEL-MEA), the reaction is carried out at a temperature in the range of 40-60° C. for 4 h.

In one of the features of the present invention, the synthetic biodegradable polymer methacrylated poly multi-carboxylic acid-polyethylene glycol (MA-PMCA-PEG) has a concentration in a range of 40-60%, w/v, and the modified natural polymer maleated gelatin (GEL-MEA) has a concentration in a range of 40-60%, w/v.

In one of the features, the present invention provides a process for fabricating a biodegradable 3D composite hydrogel scaffold comprising:
mixing of predetermined volume of a solution of MA-PMCA-PEG and a solution of GEL-MEA to obtain a polymer solution, wherein different hydrogels are obtained by varying the concentration ratio of MA-PMCA-PEG/GEL-MEA;
adding tetramethylethylenediamine (TEMED) to the polymer solution, followed by addition of a solution of ammonium persulfate (APS) and incubating till the formation ofhydrogel, and molding the hydrogel to a disc shaped 3D matrix using polypropylene mold tool to obtain the biodegradable 3D composite hydrogel scaffold.

In one of the features of the present invention, the hydrogel is prepared by adding tetramethylethylenediamine (TEMED) and a solution of ammonium persulfate (APS) to the polymer solution, followed by incubating the polymer solution.

In one of the features of the present invention, the tetramethylethylenediamine (TEMED) is added in a range of 20 to 80 ppm; the solution of ammonium persulfate is added in a range of 0.3-0.7%, w/v.

In another feature of the present invention, the volume ofMA-PMC A-PEG and GEL-MEA is in the range of 300-900 μL, the volume of TEMED is in the range of 50-70 μL, the volume of ammonium persulphate is in the range of 40-50 μL.

In yet another feature of the present invention, the incubation is carried out at a temperature in the range of 30-40° C. for 10-30 min till the formation of hydrogel.

In yet another feature of the present invention, the composite 3D hydrogel with 0.50 and 0.33 ratio of MA-PMCA-PEG/GEL-MEA exhibit swelling factor of 8.0 and 6.2 respectively.

In yet another feature of the present invention, the compression modulus of the composite 3D hydrogel with 0.50 and 0.33 ratio of MA-PMCA-PEG/GEL-MEA is 35.1 and 19.3 kPa respectively.

The present invention describes the development of cross-linkable polymers suitable for fabrication of hydrogel scaffolds. Hydrogel scaffolds are usually fabricated by cross-linking functional polymer materials using a cross-linker and initiator. In the present studly, the cross-linking moieties such as maleate or methacrylate groups have been attached on the natural polymer gelatin or synthetic polymer PMCA-PEG. The cross-linkable natural or synthetic polymers have been synthesized by simple and easily scalable techniques. The present invention provides an alternative cross-linkable gelatin for thiolated gelatin to increase its self-stability under normal atmospheric conditions. The cross-linkable polymers were cross-linked to obtain hydrogel scaffold.

In one feature, the present invention provides a synthesis of polymer PMCA-PEG by direct melt reaction of citric acid and PEG (Mw=400-2000 g/mol) using stannous chloride dihydrate as a catalyst. After the reaction, the polymer was dissolved in dimethyl formamide (DMF).

In another feature of the invention, the polymer MA-PMCA-PEG is easily scalable in a cost-economic manner using custom designed reactor.

In one feature of the present invention, the 3D composite hydrogel scaffold was fabricated to a disc shaped 3D matrix using polypropylene mold tool. The mechanical property and swelling factor of the fabricated hydrogel are disclosed. The mechanical property of the 3D composite hydrogels could be altered by changing chemical composition of MA-PMCA-PEG and GEL-MEA in the hydrogel matrix.

The present invention involves synthesis of cross-linkable synthetic and natural polymers by simple synthetic methods. The PEGDA has been widely used to make hydrogel scaffolds with natural polymers. However, the high molecular weight PEGDA is a non-degradable polymer. The linear PEGDA contains only two active sites for cross-linking, and hence the reactivity will be lesser for cross-linking. In this present invention, the cross-linkable synthetic polymer MA-PMCA-PEG exhibits multiple active sites which could be used for cross-linking with the natural polymers such as gelatin, collagen and chitosan to get three-dimensional hydrogel scaffolds. Since, the MA-PMCA-PEG is polyester, it is biodegradable and environmentally friendly to use. The MA-PMCA-PEG cross-linkable polymer is scalable in a cost-economic manner. In addition, MA-PMCA-PEG polymer could provide both cross-linking moiety and free carboxylic acid group into the hydrogel network. The cross-linking moiety could be used to fabricate hydrogel scaffold, while the free carboxylic acid group could exhibit anti-viral property.

This present invention also describes the development of cross-linkable natural polymer gelatin. In the reported works, the gelatin was widely modified with thiol groups to make hydrogel scaffold. Though the thiol based synthetic pathway of making hydrogel scaffold is a well-known and fruitful method, the commonly used thiolating agent 2-iminothiolane hydrochloride (Traut's reagent) is very expensive and hence production of bulk scale thiolated polymers is challenging. In addition, the conventional thiolated polymers are oxidized under normal atmospheric condition, involve in self cross-linking in uncontrolled manner and hence, fabrication of shape controlled hydrogel scaffold using the oxidized thiolated gelatin could not be achieved under normal atmospheric storage condition. In the present study a simple and new strategy has been disclosed to prepare cross-linkable gelatin which is highly stable under atmospheric air without undergoing self-cross-linking.

The GEL-MEA described in this present invention has been synthesized in a simple and cost effective method. The less expensive materials such as gelatin, maleic anhydride, water and acetone are only used in the present invention to synthesize GEL-MEA. Additionally, the synthesized GEL-MEA is highly stable under normal atmospheric air condition, and the cross-linking reaction using this polymer could be carried by a controlled approach. Hence, this present invention describes simple and cost effective methods for the development of cross-linkable synthetic and natural polymers. The cross-linkable polymers could be used to fabricate anti-viral composite hydrogel in industrial scale by a cost-effective approach. The mechanical properties of the fabricated hydrogel could be tuned by varying the composition of MA-PMCA-PEG and GEL-MEA in the hydrogel scaffold.

### EXAMPLES

The present disclosure with reference to the accompanying examples describes the present invention. However, those of skill in the art should, in light of the present disclosure, appreciate that many changes can be made in the specific embodiments which are disclosed and still obtain a like or similar result without departing from the spirit and scope of the invention. It is understood that the examples are provided for the purpose of illustrating the invention only and are not intended to limit the scope of the invention in any way.

### Example 1: Synthesis of Poly Multi-Carboxylic Acid-Co-Polyethylene Glycol (PMCA-PEG)

In the first step, PMCA-PEG was synthesized using citric acid and PEG by melt polymerization reaction under vacuum. The molecular weight of PEG varied from 400 to 6000 g/mol, and ratio of citric acid to PEG was varied from 2 to 10. Briefly, anhydrous citric acid (0.2 mol), PEG (0.02 mol) and stannous chloride di-hydrate (2 mmol) were mixed and heated to 110-125° C. for 1 h under vacuum (100 mm Hg). Then the reaction was carried at 140-150° C. under high vacuum (25 mm Hg) for 5 h. After cooling to room temperature, the polymer was dissolved in 100 mL of dimethyl formamide (DMF) at 70-90° C.

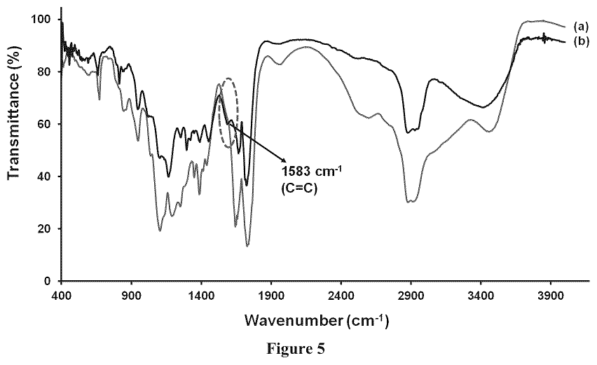

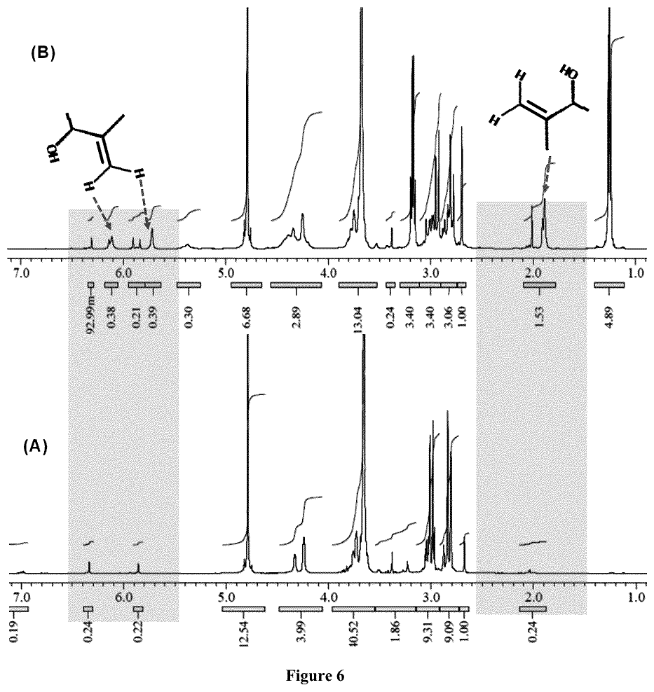

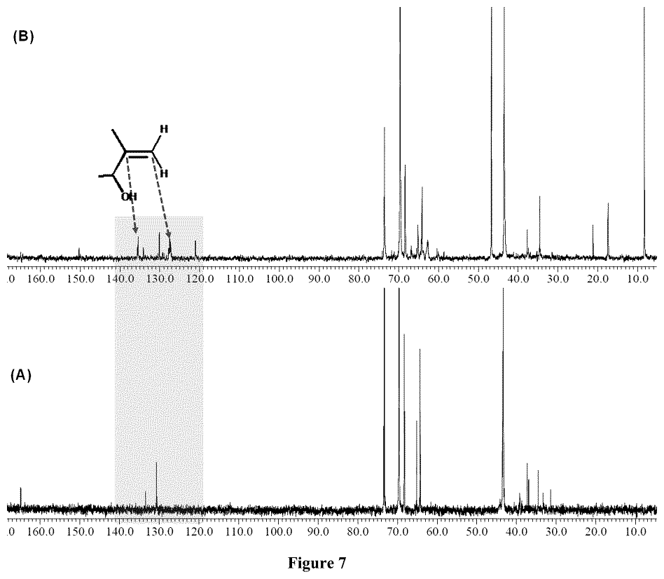

For characterization, 10 mL of this polymer solution was withdrawn and 60 mL of diethyl ether was added to precipitate the polymer and washed three times with fresh diethyl ether. The traces of DMF and diethyl ether present in the polymer product were removed on a vacuum oven at 90° C. for overnight. The PMCA-PEG was characterized by FTIR, ¹H-NMR, and ¹³C-NMR, spectra (Figure. 5-7). Yield of the product was about 70-80%.

### Example 2: Methacrylation of PMCA-PEG

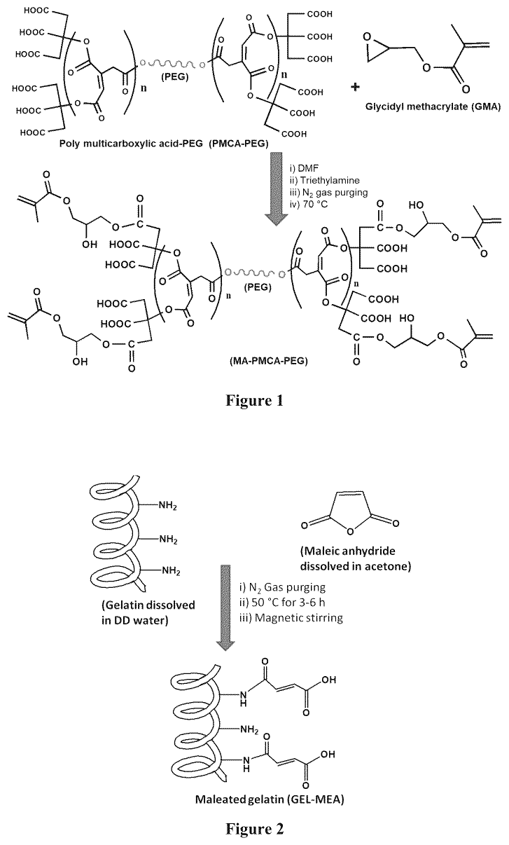

PMCA-PEG was methacrylated using glycidyl methacrylate (GMA). Briefly, to the PMC A-PEG polymer in DMF synthesized in Example 1, GMA (0.1 mol) and triethylamine (TEA) (−5 mL) were added. Then the reaction was carried at 60-80° C. for 18 h under dark and inert atmosphere with strong agitation. Finally, the MA-PMCA-PEG was recovered by adding excess diethyl ether (1-1.5 Lit), washed for three tines with diethyl ether (300-500 mL per washing). Then traces of DMF and diethyl ether present in the polymer product were removed by rotary evaporation at 80-95° C. (FIG. 1). Yield of the product was about 70%. The MA-PMCA-PEG polymer was characterized by FTIR, ¹H-NMR and ¹³C-NMR spectral techniques (Figure. 5-7).

### Example 3: Maleation of Gelatin

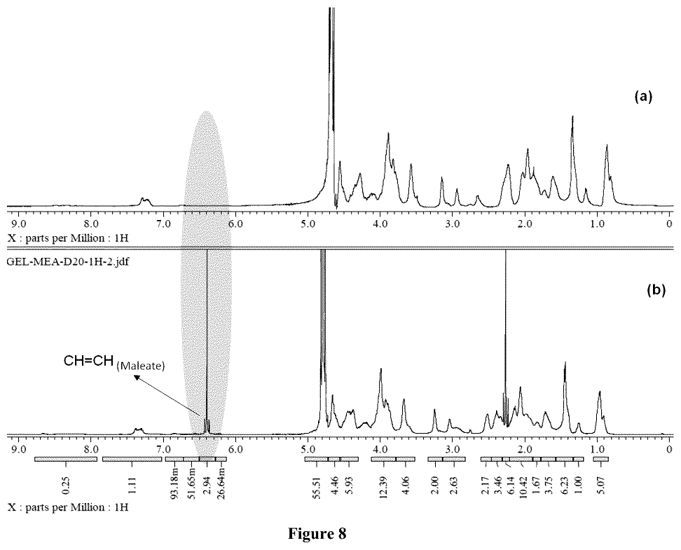

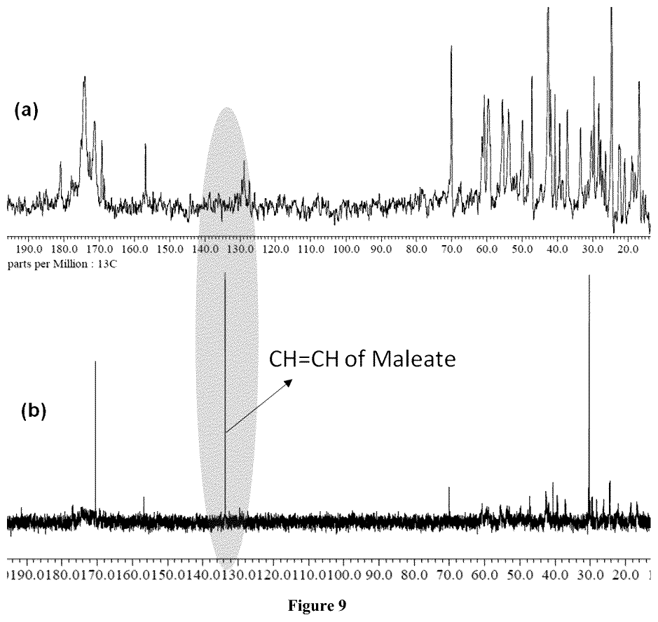

Gelatin was dissolved in de-ionized distilled water (DDW) (10%, w/v) and mixed with maleic anhydride dissolved in acetone (50%, w/v). Then, the reaction was carried out at 50° C. under nitrogen atmosphere for 4 h. After the reaction period, the GEL-MEA polymer was recovered by adding excess acetone (10 times more volumes), washed with acetone for three times and dried under vacuum for overnight (FIG. 2). The initial concentration of gelatine was not limited to 10% w/v, but the concentration was varied from 4-15% Similarly, the initial concentration of maleic anhydride in acetone was not limited to 50% w/v, but the initial concentration of maleic anhydride was varied from 25-75%. Yield of the product was about 75%. The maleation of gelatin was confirmed by H-NMR and ¹³C-NMR techniques (FIGS. 8 and 9).

### Example 4: Characterization of Polymers

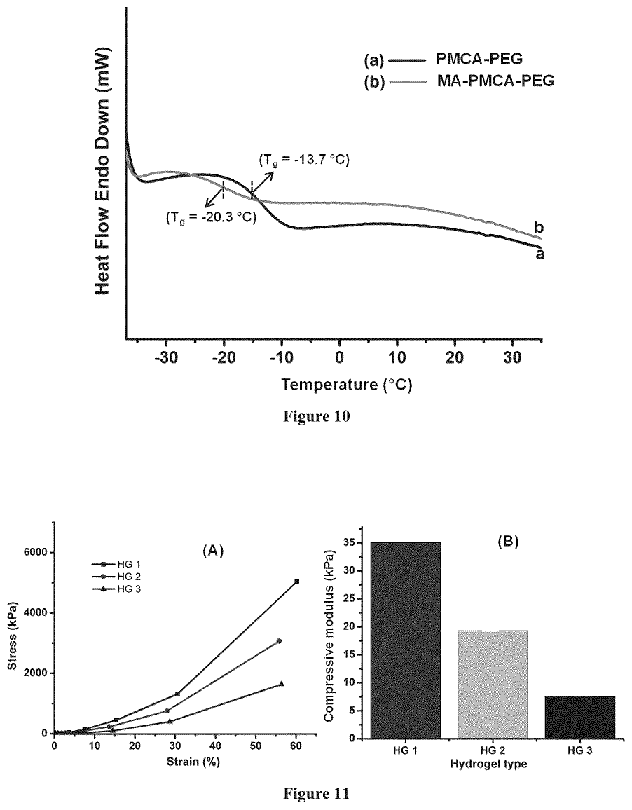

To record NMR spectral analyses, 50-100 mg of the polymer samples were dissolved in 0.6 mL of D₂O. The ¹H-NMR and ¹³C-NMR spectra of the polymer samples were recorded on a JEOL (ECA 500) NMR spectrometer operating at 500 MHz and 125.7 MHz for recording ¹H-NMR and ¹³C-NMR spectra respectively. To record FTIR spectra, 2-3 mg of polymer samples were used to make KBr pellets and the FTIR spectra were recorded on an Agilent (Carry 600) FTIR spectrometer. The differential scanning calorimetric (DSC) analyses were carried on Perkin-Elmer (Pyris) differential scanning calorimeter. The PMCA-PEG and MA-PMC A-PEG samples exhibit −13.7° C. and −20.3° C. of glass transition temperature (Tg) respectively (FIG. 10).

### Example 5: Process for Scaling Up of MA-PMCA-PEG Cross-Linkable Polymer

The MA-PMCA-PEG cross-linkable polymer was scaled up in a two stage reaction processes. In the first stage, the polymerization was carried in a custom designed 2 Lit glass reactor connected with (i) a vacuum line with liquid nitrogen moisture trap, (ii) thermal sensor, (iii) over head mechanical stirrer, and (iv) thermostat oil circulation. Initially, the citric acid (anhydrous) (1 Kg), PEG (315 g) and stannous chloride dihydrate (10 g) were transferred to the 2 Lit glass reactor. Then the reactor was heated to 125° C. at 100 mm Hg vacuum for 2 h with strong agitation. The polymerization was carried at 140-150° C. under high vacuum (25 mm Hg) for 4 h with agitation. Then, the reaction was cooled to room temperature, and the polymer product was dissolved in DMF (0.9 Lit) at 70-90° C. The vacuum line was removed from the reactor and nitrogen gas line was connected with the glass reactor via a ground joint stopper with stop cock. The entire glass reactor was covered with an aluminium foil. Triethylamine (140 mL) and glycidyl methacrylate (172.8 mL) were added. Nitrogen gas was purged for 15 min and the reaction was carried at 70° C. for 15-24 h under nitrogen and dark atmosphere.

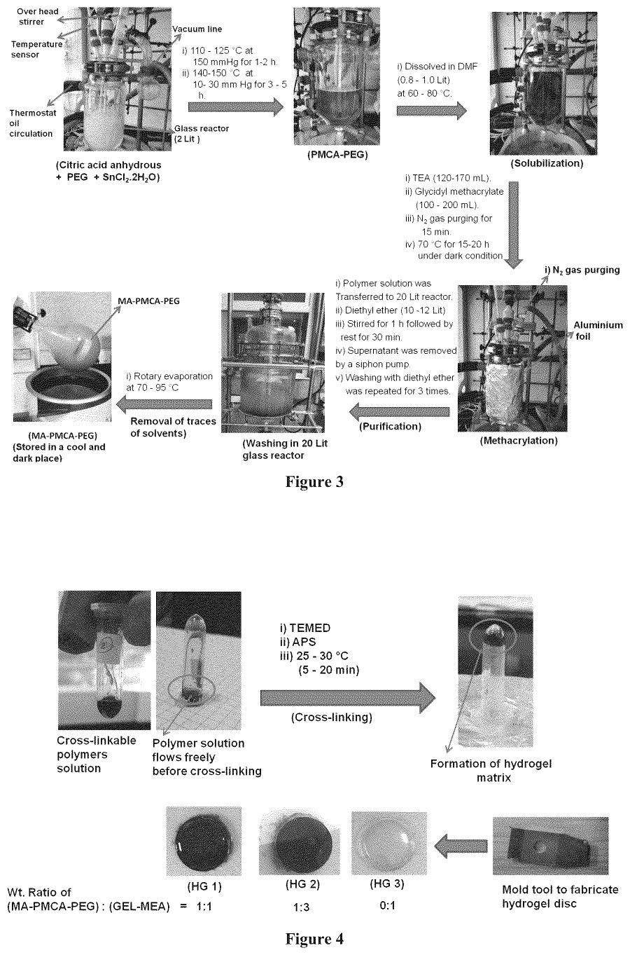

In the second stage, the polymer product in DMF was transferred to a 20 Lit glass reactor connected with (i) over head mechanical stirrer, and (ii) solvent condenser. Diethyl ether as a non-solvent (10 Lit) was added and agitated strongly for 1 h to wash out the unwanted impurities. Then, the solution was kept aside for 30 min, and the supernatant solution was drained via a siphon pump. The washing process was repeated for 3 times by adding 6 Lit of diethyl ether for 3 times. After draining the supernatant, the precipitate obtained was collected in a 3 Lit RB flask and the remaining solvents were removed by rotary evaporation at 95° C. Finally, the obtained MA-PMCA-PEG was stored under dark and cool condition (FIG. 3). (Yield: 80%)

### Example 6: Fabrication of Hydrogel Scaffold

To fabricate hydrogel scaffold, the cross linkable polymers were cross-linked with an initiator in the presence of tetramethylethylenediamine (TEMED). The MA-PMCA-PEG (50%, w/v) and GEL-MEA (50%, w/v), ammonium persulfate (APS) (10%, w/v) were prepared in deaerated water. The predetermined volumes (300-900 μL) of MA-PMCA-PEG and GEL-MEA were mixed together to get different hydrogels with various concentration of GEL-MEA and MA-PMCA-PEG (Table 1). Then, 60 μL of TEMED was added to the mixture of polymer solutions, followed by 45 μL of APS (effective concentration of 0.45%) was added and incubated at 30-40° C. After 10-30 min, formation of hydrogel was observed (FIG. 4). The concentration of MA-PMCA-PEG and GEL-MEA was not restricted to 50%, but it varied from 40-60% w/v.

### Example 7: Determination of Swelling Factor of Hydrogels

To determine the swelling factor, the fabricated hydrogels were dried in a hot air oven at 40° C. overnight. After drying, the cross-linked polymer scaffold was immersed in 5 mL of phosphate buffer (pH 7.4) and incubated at 25° C. for 4 h. Then, weight of the swelled gels was measured after carefully taking from phosphate buffer. The swelling factor of the hydrogels was calculated by using the following formula,

$$Swelling factor = ( {M}_{t} - {M}_{i} ) / {M}_{i}$$

Where, Mᵢ denotes initial weight of dried polymer before swelling, and Mₜ denotes weight of swelled gel.

The gelatine based 3D hydrogel with 0.5 and 0.33 ratio of MA-PMCA-PEG/GEL-MEA exhibit swelling factor of 8.0 and 6.2 respectively (Table 1).

### Example 8: Compression Modulus of Hydrogels

To measure the compression modulus, different composition of hydrogel discs (Table 1) with a diameter of 8 mm and a height of 3-3.5 mm were fabricated using a polypropylene mold tool. Stress-Strain measurements were carried on a TA ARES-G2 Rheometer equipped with a 2.0 Kg load cell. The hydrogel discs were compressed at a rate of 0.005 mm/s using a probe diameter of 40 mm. Stress-strain curves were plotted and compressive modulus of different hydrogel discs was determined as the slope of linear regression in the range of 3 to 15% of strain (FIG. 11 and Table 1). The compressive modulus of 3D composite hydrogel increases with increasing amount of MA-PMCA-PEG in the hydrogel scaffold. The compression modulus of the 3D hydrogel with 0.5 and 0.33 ratio of MA-PMC A-PEG/GEL-MEA is 35.1 and 19.3 kPa respectively (Table 1).

TABLE 1
Fabrication and properties of hydrogel matrices with different
composition of MA-PMCA-PEG and GEL-MEA.

| Sl. | Hydrogel | Ratio of MA-PMCA- | Swelling | Compression |
|---|---|---|---|---|
| No | type | PEG/GEL-MEA | factor | modulus (kPa) |
| 1 | HG 1 | 0.5 | 8.0 | 35.1 |
| 2 | HG 2 | 0.33 | 6.2 | 19.3 |
| 3 | HG 3 | 0.0 | 7.9 | 7.6 |

We claim:

1. A biodegradable hydrogel for 3D composite hydrogel scaffold comprising:
a synthetic biodegradable polymer methacrylated poly multi-carboxylic acid-polyethylene glycol (MA-PMCA-PEG); and
a modified natural polymer maleated gelatin (GEL-MEA).

2. The biodegradable hydrogel of claim 1, wherein the synthetic biodegradable polymer methacrylated poly multi-carboxylic acid-polyethylene glycol (MA-PMCA-PEG) to the modified natural polymer maleated gelatin (GEL-MEA) has a ratio in a range of 0.33 to 0.5.

3. The biodegradable hydrogel of claim 1, wherein the synthetic biodegradable polymer methacrylated poly multi-carboxylic acid-polyethylene glycol (MA-PMCA-PEG) to the modified natural polymer maleated gelatin (GEL-MEA) has a ratio of 0.5 exhibit a swelling factor of 8.0 and a compression modulus of 35.1 kPa.

4. The biodegradable hydrogel of claim 1, wherein the synthetic biodegradable polymer methacrylated poly multi-carboxylic acid-polyethylene glycol (MA-PMCA-PEG) to the modified natural polymer maleated gelatin (GEL-MEA) has a ratio of 0.33 exhibit the swelling factor of 6.2, and the compression modulus of 19.3 kPa.

5. The biodegradable hydrogel of claim 1, for use in preparing a biodegradable 3D composite hydrogel scaffold.

6. A synthetic biodegradable cross linkable polymer:
methacrylated poly multi-carboxylic acid-polyethylene glycol (MA-PMCA-PEG)

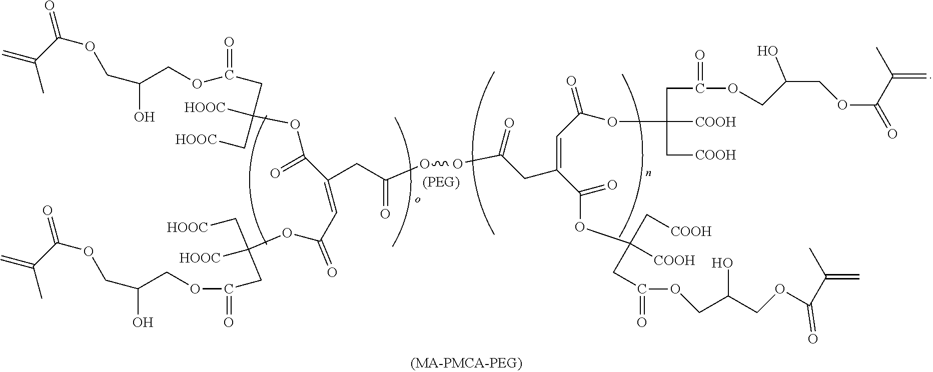

7. A process for fabricating a biodegradable 3D composite hydrogel scaffold comprising:
preparing a synthetic biodegradable polymer methacrylated poly multi-carboxylic acid-polyethylene glycol (MA-PMCA-PEG);
preparing a modified natural polymer maleated gelatin (GEL-MEA);
mixing of predetermined volume of a solution of the synthetic biodegradable polymer methacrylated poly multi-carboxylic acid-polyethylene glycol (MA-PMCA-PEG) and a solution of the modified natural polymer maleated gelatin (GEL-MEA) to obtain a polymer solution; and
preparing a hydrogel from the polymer solution and molding the hydrogel to obtain the biodegradable 3D composite hydrogel scaffold,
wherein different hydrogels are obtained by varying the ratio of (MA-PMC A-PEG) and (GEL-MEA).

8. The process of claim 7, wherein the synthetic biodegradable polymer methacrylated poly multi-carboxylic acid-polyethylene glycol (MA-PMCA-PEG) is prepared by a process comprising reacting poly multi-carboxylic acid-polyethylene glycol (PMCA-PEG) polymer in dimethylformamide (DMF) with glycidyl methacrylate (GMA) in the presence of triethylamine (TEA) and adding diethyl ether to recover MA-PMCA-PEG.

9. The process of claim 8, wherein, the glycidyl methacrylate (GMA) is added in a range of 0.02-0.1 mol, and the triethylamine is added in a range of 5-15 mL.

10. The process of claim 8, wherein the reaction is carried out at a temperature in a range of 60-80° C. for 15 to 24 h under dark and inert atmosphere with agitation and the solvent is dimethyl formamide.

11. The process of claim 7, wherein the modified natural polymer maleated gelatin (GEL-MEA) is prepared by a process comprising dissolving gelatin in water to obtain a gelatin solution and reacting the gelatin solution with a maleating agent in acetone to obtain the modified natural polymer maleated gelatin (GEL-MEA).

12. The process of claim 11, wherein the maleating agent is maleic anhydride; the amount of gelatin in water is in a range of 5-15%, w/v; and the amount of maleic anhydride in acetone is in a range of 40-60%, w/v.

13. The process of claim 11, wherein the reaction is carried out at a temperature in a range of 40-60° C. for 4 h.

14. The process of claim 7, wherein the synthetic biodegradable polymer methacrylated poly multi-carboxylic acid-polyethylene glycol (MA-PMCA-PEG) has a concentration in a range of 40-60%, w/v, and the modified natural polymer maleated gelatin (GEL-MEA) has a concentration in a range of 40-60%, w/v.

15. The process of claim 7, wherein the hydrogel is prepared by adding tetramethylethylenediamine (TEMED) and a solution of ammonium persulfate (APS) to the polymer solution, followed by incubating the polymer solution.

16. The process of claim 15, wherein the tetramethylethylenediamine (TEMED) is added in a range of 20 to 80 ppm; the solution of ammonium persulfate is added in a range of 0.3-0.7%, w/v.

17. The process of claim 15, wherein the incubation is carried out at a temperature in a range of 30-40° C. for 10-30 min.

18. The process of claim 7, wherein the hydrogel is molded into a disc shaped 3D matrix using a mold tool to obtain the biodegradable 3D composite hydrogel scaffold.
